(19) United States
(12) Patent Application Publication
(10) Pub. No.: US 2023/0154575 A1
(43) Pub. Date: May 18, 2023

(54) Systems and Methods for Mental Health Care Delivery Via Artificial Intelligence

(71) Applicant: HealMed Solutions LLC, New York, NY (US)

(72) Inventors: Nidhi GOEL, New York, NY (US); Bhoopathi RAPOLU, Fishers, IN (US)

(21) Appl. No.: 17/919,569

(22) PCT Filed: Jun. 3, 2021
(86) PCT No.: PCT/US2021/035588

§ 371 Date: Oct. 18, 2022

Related U.S. Application Data
(60) Provisional application No. 63/034,568, filed on Jun. 4, 2020.

(51) Int. Cl. G16H 10/20; G16H 50/30; G16H 50/20

(52) U.S. Cl. CPC G16H 10/20; G16H 50/30; G16H 50/20

(57) ABSTRACT

The present disclosure provides systems, methods, and compositions for mental health analysis, such as obtaining a mental health intake questionnaire from a subject and reporting the analysis of the mental health questionnaire to a medical professional.

### CROSS REFERENCE TO RELATED APPLICATIONS

This application claims the benefit of U.S. Provisional Application No. 63/034,568 filed Jun. 4, 2020, the disclosure of which is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

A large number of people need access to mental health care. However, there may be limited resources available to people (e.g., patients) who require meetings with a mental health professional. For example, there is a U.S. mental health crisis due to a shortage of psychiatrists, with about half of countries having no psychiatrists and 49 states having a severe shortage. Further, 1 in every 5 people are suffering with mental health issues. Providing quality mental health care in a timely manner is important, but low resources and stressed staff may make this difficult to achieve.

### SUMMARY

Recognizing a need to address a shortage of behavioral health care providers, the present disclosure provides systems, methods, and compositions for mental health analysis, including reporting an analysis of a mental health questionnaire to a medical professional. For example, an adaptable questionnaire that can be given at a primary health clinic can provide physicians and mental health professionals with a valuable resource for identifying underlying problems. The questionnaire can identify potential mental health issues while decreasing the time necessary to administer a questionnaire in person and identify potential issues.

In an aspect, the present disclosure provides a system comprising: a computing device comprising a memory, one or more executable instructions, one or more processors operably connected to a memory, wherein one or more processors can be configured to execute one or more executable instructions to perform at least: (a) retrieving a medical questionnaire for a subject; (b) generating an adaptable medical questionnaire comprising a series of one or more medical questions to be presented to a subject, wherein subsequent questions of a series of one or more medical questions can be selected by an artificial intelligence (AI)-based algorithm based at least in part on a previous response of a subject to one or more prior questions of a series of one or more medical questions; (c) presenting an adaptable medical questionnaire to a subject; (d) receiving data of a series of one or more responses from a subject, responsive to a presenting; and (e) processing a data to transcribe a series of one or more responses from a subject into a plurality of subject intents, wherein at least one of a processing and selecting subsequent questions can be performed in real-time.

In some embodiments, the computing device can be a mobile device. In some embodiments, the mobile device can comprise a tablet computer, a laptop computer, a smartphone, or a smartwatch. In some embodiments, the system can further comprise a visual headset operably connected to one or more processors. In some embodiments, the visual headset can be configured to provide a virtual reality (VR) or augmented reality (AR) experience to a subject. In some embodiments, the system can further comprise a camera operably connected to one or more processors. In some embodiments, the camera can comprise a video camera. In some embodiments, the system can further comprise a graphical user interface (GUI). In some embodiments, the GUI can comprise a personal computing operating system. In some embodiments, the personal computing operating system can comprise a mobile device operating system. In some embodiments, the GUI can comprise a virtual reality environment, a hologram, or any combination thereof. In some embodiments, the GUI can be configured to perform at least one of: presenting an adaptable medical questionnaire to a subject and providing a plurality of subject intents to a health care professional. In some embodiments, the GUI can be configured to present an adaptable medical questionnaire to a subject. In some embodiments, the GUI can comprise a first user interface to be used by a subject. In some embodiments, the GUI can be configured to provide a plurality of subject intents to a health care professional. In some embodiments, the GUI can comprise a second user interface to be used by a health care professional. In some embodiments, the graphical user interface of a healthcare professional can be different from a graphical user interface of a subject.

In some embodiments, the adaptable medical questionnaire can be administered to a subject to be answered prior to or during a health care visit of a subject. In some embodiments, the adaptable medical questionnaire can be administered via one or more of auditory intake and visual intake. In some embodiments, the adaptable medical questionnaire can be administered via an auditory intake and a visual intake. In some embodiments, the system can further comprise a microphone or a speaker operably connected to one or more processors. In some embodiments, the system can further comprise a microphone and a speaker operably connected to one or more processors. In some embodiments, the adaptable medical questionnaire can comprise a series of one or more auditory intake medical questions that can be administered through a speaker. In some embodiments, the adaptable medical questionnaire can comprise a series of one or more auditory intake medical questions that can be responded to through a microphone. In some embodiments, the series of one or more auditory intake medical questions can be administered at least in part in English. In some embodiments, the series of one or more auditory intake medical questions can be administered in a language primarily other than English. In some embodiments, the series of one or more auditory intake medical questions can be administered at least in part in a language selected from: Spanish, Mandarin Chinese, Hindi, Arabic, German, and French. In some embodiments, the series of one or more auditory intake medical questions can comprise an option to select a language from among a plurality of languages for administering a series of one or more auditory intake medical questions. In some embodiments, the adaptable medical questionnaire can comprise a series of one or more visual intake medical questions. In some embodiments, the series of one or more visual intake medical questions can be administered at least in part in English. In some embodiments, the series of one or more visual intake medical questions can be administered in a language primarily other than English. In some embodiments, the series of one or more visual intake medical questions can be administered at least in part in a language selected from: Spanish, Mandarin Chinese, Hindi, Arabic, German, and French. In some embodiments, the series of one or more visual intake medical questions can comprise visual pictures that can provide information about a medical question to a subject. In some embodiments, the series of one or more visual intake medical questions can comprise an option to select a language from among a plurality of languages for administering a series of one or more visual intake medical questions. In some embodiments, auditory intake medical questions and visual intake medical questions can be linked. In some embodiments, the medical questionnaire can be retrieved from a database. In some embodiments, the database can comprise a cloud-based network. In some embodiments, the series of one or more medical questions can comprise medical questions for assessing mental health. In some embodiments, the medical questionnaire can be for at least in part obtaining general subject information. In some embodiments, the medical questionnaire can be for at least in part for assessing general health. In some embodiments, the medical questionnaire can comprise medical questions derived from American Psychological Association (APA) guidelines. In some embodiments, the database can comprise at least about 10, about 50, about 100, about 200, about 300, about 400, about 500, about 600, about 700, about 800, about 900, about 1000, about 1500, about 2000, about 2500, or about 3000 medical questions. In some embodiments, the medical questions can comprise binary or open-ended questions. In some embodiments, the medical questions can comprise binary questions corresponding to “yes” or “no” responses from a subject. In some embodiments, the medical questions can comprise open-ended questions corresponding to open-ended responses from a subject. In some embodiments, the binary or open-ended questions may correspond to a non-response from a subject. In some embodiments, the adaptable medical questionnaire can be administered remotely to a subject through a communications network.

In some embodiments, the subject can be a patient. In some embodiments, the patient can have a disease or disorder or may be suspected of having a disease or disorder. In some embodiments, the health care visit may be performed at a clinic, an emergency room, a primary care facility, an urgent care facility, a mental health facility, a psychiatric ward, a hospital, a detention facility, or any combination thereof. In some embodiments, the health care visit can comprise: a remote visit, a telemedicine visit, a virtual visit, or any combination thereof.

In some embodiments, the subsequent questions can be selected from a database. In some embodiments, the subsequent questions can comprise questions generated by an AI-based algorithm. In some embodiments, the AI-based algorithm can comprise: a linear regression, a logistic regression, a Natural Language Processing (NLP), a Bayesian network, a maximum entropy, a conditional random field, a Random Forest, a support vector machine (SVM), a neural network, a deep learning algorithm, or a combination thereof. In some embodiments, the AI-based algorithm can be programmed to determine a subject intent based on one or more responses from a subject. In some embodiments, the AI-based algorithm can be programmed to perform a logic. In some embodiments, the AI-based algorithm can be programmed to analyze words and sentence structure of one or more responses from a subject to at least in part estimate an education level of a subject. In some embodiments, the AI-based algorithm can be programmed to determine one or more potential health issues of a subject. In some embodiments, the AI-based algorithm can be programmed to determine a patient risk of one or more potential health issues. In some embodiments, the patient risk can be determined from among a plurality of different patient risk categories. In some embodiments, the plurality of different patient risk categories can comprise at least two of: critical, alarming, abnormal, and normal. In some embodiments, the plurality of different patient risk categories can comprise critical and alarming, wherein critical and alarming can be communicated to a healthcare professional. In some embodiments, the data can comprise a visual data, an audio data, or any combination thereof. In some embodiments, the transcribing can be performed at least in part in English. In some embodiments, the transcribing can be performed in a language primarily other than English. In some embodiments, the transcribing can be performed at least in part in a language selected from: Spanish, Mandarin Chinese, Hindi, Arabic, German, and French. In some embodiments, the one or more responses from a subject can be translated at least in part to English prior to transcribing. In some embodiments, the one or more responses from a subject can be translated at least in part to a language primarily other than English prior to transcribing. In some embodiments, the one or more responses from a subject can be translated at least in part to a language selected from: Spanish, Mandarin Chinese, Hindi, Arabic, German, and French prior to transcribing. In some embodiments, transcribing the series of one or more responses from a subject into a plurality of subject intents can comprise selecting a subject intent from among a plurality of subject intents. In some embodiments, the plurality of subject intents can comprise binary subject intents.

In some embodiments, performing in real-time can comprise transmitting through a network a data and a plurality of subject intents to a second computing device. In some embodiments, performing in real-time can comprise transmitting through a network a data or a plurality of subject intents to a second computing device. In some embodiments, the second computing device can comprise a server, a cloud-based server, a personal computer, a smart mobile device, or any combination thereof. In some embodiments, the network can comprise a wired or a wireless network. In some embodiments, the data and the plurality of subject intents can be accessible by a health care professional during an administering of an adaptable medical questionnaire to a subject. In some embodiments, the data or the plurality of subject intents can be accessible by a health care professional during an administering of an adaptable medical questionnaire to a subject. In some embodiments, the plurality of subject intents can be provided in a database format that can be accessible by a health care professional. In some embodiments, the database format can comprise an electronic health record format.

In some embodiments, the one or more processors can be configured to execute one or more executable instructions to further perform identifying a set of one or more potential health issues of a subject based at least in part on a plurality of subject intents. In some embodiments, the one or more processors can be configured to execute one or more executable instructions to further provide a set of one or more potential health issues of a subject to a health care professional. In some embodiments, the plurality of subject intents can be provided to a health care professional through a GUI. In some embodiments, the one or more processors can be configured to execute one or more executable instructions to further generate a diagnosis or an indication of a subject having a disease or disorder or being at elevated risk for a disease or disorder. In some embodiments, the disease or disorder can comprise a substance-related and addictive disorder, a non-substance-related disorder, an anxiety disorder, an attention deficit disorder, an attention hyperactivity disorder, bipolar and related disorders, a depressive disorders, an eating disorder, obsessive compulsive and related disorder, schizophrenia spectrum and other psychotic disorders, dissociative disorders, somatic-symptom and related disorders, sexual disorders, sleep-wake disorders, personality disorders, trauma and stress disorders, neuro-cognitive disorders, disruptive impulse-control and conduct disorders, other mental disorders or any combination thereof. In some embodiments, the set of one or more potential health issues can comprise: a substance-related and addictive disorder, a non-substance-related disorder, an anxiety disorder, an attention deficit disorder, an attention hyperactivity disorder, bipolar and related disorders, a depressive disorders, an eating disorder, obsessive compulsive and related disorder, schizophrenia spectrum and other psychotic disorders, dissociative disorders, somatic-symptom and related disorders, sexual disorders, sleep-wake disorders, personality disorders, trauma and stress disorders, neuro-cognitive disorders, disruptive impulse-control and conduct disorders, other mental disorders or any combination thereof. In some embodiments, at least one of a set of one or more potential health issues and a plurality of subjects intents can be recorded in an electronic medical record of a subject. In some embodiments, at least one of a data and a plurality of subject intents can be communicated through a communications medium. In some embodiments, the healthcare professional can comprise a medical doctor, a registered nurse, a physician's assistant, a nurse practitioner, a doctor of osteopathic medicine, a psychiatrist, a psychologist, a social worker, a mental health therapist, a mental health counselor or any combination thereof.

In another aspect, the present disclosure provides a computer-implemented method, comprising: (a) retrieving a medical questionnaire for a subject; (b) generating an adaptable medical questionnaire comprising a series of one or more medical questions to be presented to a subject, wherein subsequent questions of the series of one or more medical questions can be selected by an artificial intelligence (AI)-based algorithm based at least in part on a previous response of a subject to one or more prior questions of the series of one or more medical questions; (c) presenting the adaptable medical questionnaire to the subject; (d) receiving data of a series of one or more responses from the subject, responsive to the presenting; and (e) processing the data to transcribe the series of one or more responses from the subject into a plurality of subject intents, wherein at least one of the processing and selecting the subsequent questions can be performed in real-time.

In another aspect, the present disclosure provides a non-transitory computer storage medium storing instructions that can be operable when executed by one or more processors to implement a method comprising: (a) retrieving a medical questionnaire for a subject; (b) generating an adaptable medical questionnaire comprising a series of one or more medical questions to be presented to a subject, wherein subsequent questions of a series of one or more medical questions can be selected by an artificial intelligence (AI)-based algorithm based at least in part on a previous response of a subject to one or more prior questions of a series of one or more medical questions; (c) presenting an adaptable medical questionnaire to a subject; (d) receiving data of a series of one or more responses from a subject, responsive to a presenting; and (e) processing data to transcribe a series of one or more responses from a subject into a plurality of subject intents, wherein at least one of a processing and selecting subsequent questions can be performed in real-time.

In another aspect, the present disclosure provides a health care facility comprising a system of the present disclosure.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a method 100 of the present disclosure.

FIG. 2 shows an example of a screen shot from the application of a patient's user interface.

FIG. 3 shows an example of a screen shot from the application of an initial care provider (ICP) user interface.

FIG. 4 shows an example of a screen shot from the application of a health care provider's user interface.

FIG. 5 shows an example of a screen shot from the application of a hospital administrator's user interface.

FIG. 6 shows a computer system that is programmed or otherwise configured to implement systems and methods provided herein.

FIG. 7 shows an example of a screen shot from the system assessment report application of a health care provider's user interface.

FIG. 8 shows an example of a screen shot from the mental status exam application of a health care provider's user interface.

FIG. 9 shows an example of a screen shot from the treatment plan application of a health care provider's user interface.

FIG. 10 shows an example of a screen shot from the application of a health care provider's user interface.

FIG. 11 shows an examples of a screen shots from the system assessment report application of a health care provider's user interface.

FIG. 12 shows an example of a screen shot from the system assessment report application of a health care provider's user interface.

FIG. 13 shows an example of a screen shot from the mental status examination application of a health care provider's user interface.

FIG. 14 shows an example of a screen shot of a video recording of a subject from the health care provider's user interface.

FIG. 15 shows an example of a screen shot from the treatment plan application of a health care provider's user interface.

FIG. 16 shows an example of screen shots from the medical decision making application of a health care provider's user interface.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a,” “an,” and “the” include plural references, unless the context clearly dictates otherwise. For example, the term “a question” includes a plurality of questions.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the term “subject,” generally refers to an organism (e.g., a human) that can answer a questionnaire that comprises questions that relate to general information, general health, mental health, or any combination thereof. A subject may be a person (e.g., an individual or a patient). The subject may be male or female. The subject may be a child, or an adult. The subject can be about 1 year old to about 18 years old. The subject can be about 5, 10, 15, 20, 25, 30, 35, 40, 45, 50, 55, 60, 65, 70, 75, 80, 85, 90, 95, 100, 105, 110, 115, 120, 125 or 130 years old. The subject may be a vertebrate, such as, for example, a mammal. Non-limiting examples of mammals include humans, simians, farm animals, sport animals, and pets. The subject may be a non-human. In some cases, the human may answer the question on behalf of a non-human subject (e.g., a simian, a farm animal, a sport animal, or a pet). The subject may have or be suspected of having a health or physiological condition, such as a disease or mental illness. The subject may be healthy. In some examples, the subject is a patient. As an alternative, the subject may be asymptomatic with respect to the health or physiological condition (e.g., disease).

Mental Health Analysis

A large number of people need access to mental health care. However, there may be limited resources available to people (e.g., patients) who require meetings with a mental health professional. For example, there is a U.S. mental health crisis due to a shortage of psychiatrists, with about half of countries having no psychiatrists and 49 states having a severe shortage. Further, 1 in every 5 people are suffering with mental health issues. Providing quality mental health care in a timely manner is important, but low resources and stressed staff may make this difficult to achieve.

Recognizing a need to address a shortage of behavioral health care providers, the present disclosure provides systems, methods, and compositions for mental health analysis, including reporting an analysis of a mental health questionnaire to a medical professional. For example, an adaptable questionnaire that can be given at a primary health clinic can provide physicians and mental health professionals with a valuable resource for identifying underlying problems. The questionnaire can identify potential mental health issues while decreasing the time necessary to administer a questionnaire in person and identify potential issues.

Using systems and methods of the present disclosure, the effectiveness of mental health care services may be improved by leveraging a voice-activated AI platform. Solutions can be provided to the daily struggles faced by behavioral health care providers and patients, and the efficiency and effectiveness of psychiatric service delivery may be improved. Models may be developed and tested for data-driven triage, treatment, and decision-making and outcomes. For example, if a patient may have suicidal thoughts, the AI can alert a healthcare professional during the administration of the questionnaire.

Currently, there is an acute shortage of psychiatrists shifting access for behavioral health to emergency departments (about 40% of emergency department visits are for behavioral health issues). Emergency departments are ill equipped due to low numbers of specialist staff for screening and providing proper care to this unique population. The average wait time for behavioral health patients in emergency departments can range from 6 hours to 6 days. Psychiatrists are overburdened with high caseloads and excessive time spent on charting in electronic medical records, leaving limited time to provide clinical care.

Behavioral health care delivery may face challenges, such as geographical barriers and delays to receive an appointment, and limited time with the health care provider. This can lead to an unsatisfying experience for the patient, and the health care provider increases the likelihood of poor compliance to treatment, medical errors, continued morbidity, or a combination thereof.

From a psychiatrist's perspective, charting burden, poor quality electronic medical records (EMR), and cramped schedules are increasing the burn-out rate of psychiatrists. Documentation and policy burdens on physicians can hamper their ability to provide behavioral care to a higher volume of patients. For example, 55% of psychiatrists may be at least 55 years or older, and not well-versed with computers and EMR systems. Implementation of electronic medical records as industry standard may be a challenge for providers.

Using systems and methods of the present disclosure, a digital technology platform utilizing AI can offer solutions to assist health care providers to improve the delivery of mental health care, thereby increasing efficacy, effectiveness, and consumer and provider satisfaction. For example, one or more of the following advantages may be realized using systems and methods of the present disclosure: improved ability to deliver evidence-based psychiatric services in a timely manner, a platform that facilitates telemedicine consulting thereby reducing wait times to see a health care provider regardless of the care setting, increased efficiency of emergency departments by providing timely intake and triage, minimizing charting time for providers thereby reducing EMR errors, being developed by an ABPN double board certified psychiatrist with extensive clinical and administrative experience, use of semi-structured clinical interviews for patient intake and screening on a portable handheld device, evidence showing that patients are more likely to be open discussing sensitive personal items via AI virtual support, and a platform that features simulated and understandable artificial emotional intelligence (AEI), visual, and verbal cues to help judge human emotional states.

Using systems and methods of the present disclosure, a digital technology platform utilizing AI may be designed to improve the consumer and provider experience by assisting delivery of psychiatric care for facilities without an attending psychiatrist, serve as a helpful tool to primary care providers to reach a most probable diagnosis (Clinical Decision Support System), and allow a psychiatrist to provide care for multiple facilities simultaneously. For example, health care providers at large institutions, such as schools and colleges, may use the digital technology platform to screen moderate to high-risk students on a periodic basis to provide timely intervention.

A large number of diseases or disorders may arise at least in part because of a mental health illness. Thus, analysis of mental health intake questionnaire data of subjects may provide valuable insights into disease causes and risks as well as lifestyle recommendations for a subject to manage his or her own health. However, health intake questionnaires may generate complex datasets that are difficult to interpret and understand by an end user, such as a subject or patient. Mental health data generated from one or more questionnaires may need to be efficiently collected, analyzed, and interpreted to understand potential mental health issues. Thus, there may be a need for fast and effective reporting of mental health data that is comprehensive, clinically tested, and easy to understand. The adaptable questionnaire described herein, may provide significant improvements in at least the processing of patients, identifying a potential issue, compiling patient data, and time spent with a patient addressing an identified problem.

In some embodiments, the present disclosure provides a computer-implemented method for generating a mental health profile of a subject. The method may comprise administering a mental health questionnaire to the subject comprising a question or a plurality of questions, wherein the mental health information is obtained by processing the responses obtained or derived from the subject using the questionnaire. The mental health information can be processed to identify one or more mental health diseases, disorders or conditions of the subject. One or more mental health profiles can be identified and can correspond to a mental disease. In some cases a mental disease can comprise substance-related and addictive disorder, a non-substance-related disorder, an anxiety disorder, an attention-deficit disorder, an attention hyperactivity disorder, bipolar and related disorders, a depressive disorder, an eating disorder, obsessive compulsive and related disorders, schizophrenia spectrum and other psychotic disorders, dissociative disorders, somatic-symptom and related disorders, a sexual disorder, a sleep-wake disorder, a personality disorder, a trauma and stress disorder, a neuro-cognitive disorder, a disruptive impulse-control and conduct disorder, another mental disorder or any combination thereof.

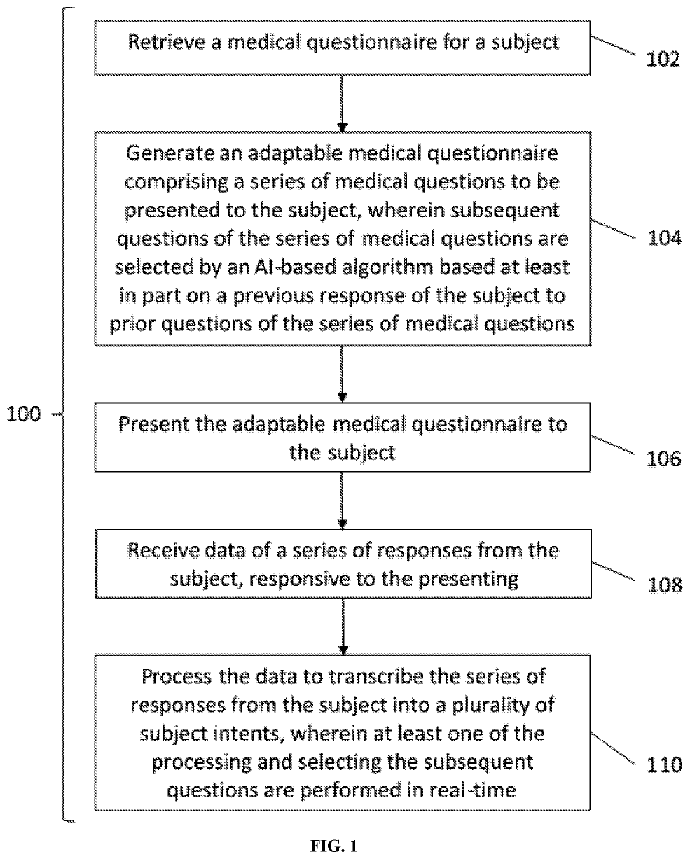

FIG. 1 shows an example of a method 100 of the present disclosure. The method 100 may comprise retrieving a medical questionnaire for a subject (as in operation 102). Next, the method 100 may comprise generating an adaptable medical questionnaire comprising a series of one or more medical questions to be presented to the subject, wherein subsequent questions of the series of one or more medical questions can be selected by an artificial intelligence (AI)-based algorithm based at least in part on a previous response of the subject to one or more prior questions of the series of one or more medical questions (as in operation 104). Next, the method 100 may comprise presenting the adaptable medical questionnaire to the subject (as in operation 106). Next, the method 100 may comprise receiving data of a series of one or more responses from the subject, responsive to the presenting (as in operation 108). Next, the method 100 may comprise processing the data to transcribe the series of one or more responses from the subject into a plurality of subject intents, wherein at least one of the processing and selecting the subsequent questions can be performed in real-time (as in operation 110).

The term “computing device” as used herein, generally refers to a device that can comprise a memory, one or more executable instructions, one or more processors operably connected to the memory, wherein the one or more processors are configured to execute the one or more executable instructions. In some cases, a computing device can be a mobile device, a stationary device or any combination thereof. In some instances, a mobile device can be a tablet computer, a laptop computer, a smartphone, a mobile smart device, a virtual reality device, smart glass, smart glasses, a smartwatch, or any combination thereof. In some instance, a computing device can be a desktop personal computer, a gaming system, a smart TV, a smart speaker (e.g., Amazon Echo™), a smart device, or any combination thereof. In some cases, a computing device can be connected to the internet, a private network, a secure network or any combination thereof. In some cases, a computing device can be operably connected to a speaker, visual headset, a video display, a projector, a touch screen, a microphone, a camera, a video camera, a printer, a scanner, a fax machine, an input device (e.g., a keyboard or a mouse), or any combination thereof. A speaker can be an integrated speaker, an auxiliary speaker (e.g., headphones or a standalone speaker system), or any combination thereof. A microphone can be an integrated microphone, an auxiliary microphone or any combination thereof. In some instances, a speaker and a microphone can be combined into an operable unit. In some cases, a visual headset can be configured to provide a virtual reality or augmented reality experience to the subject.

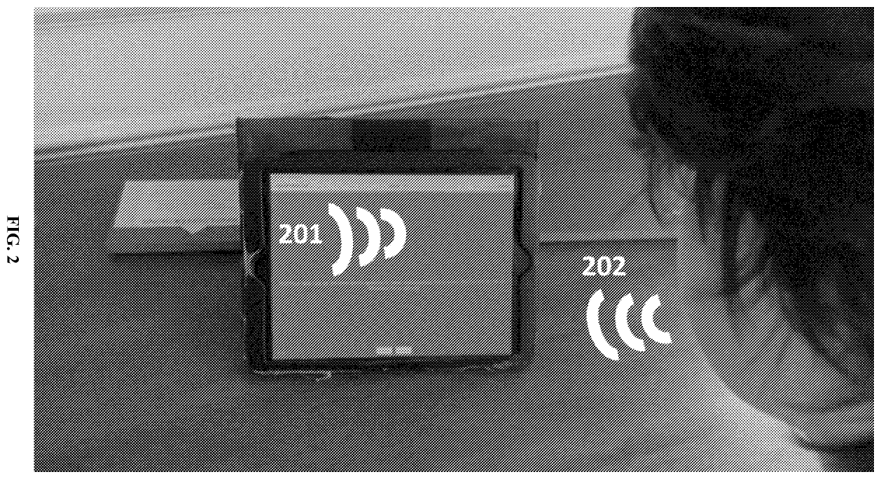

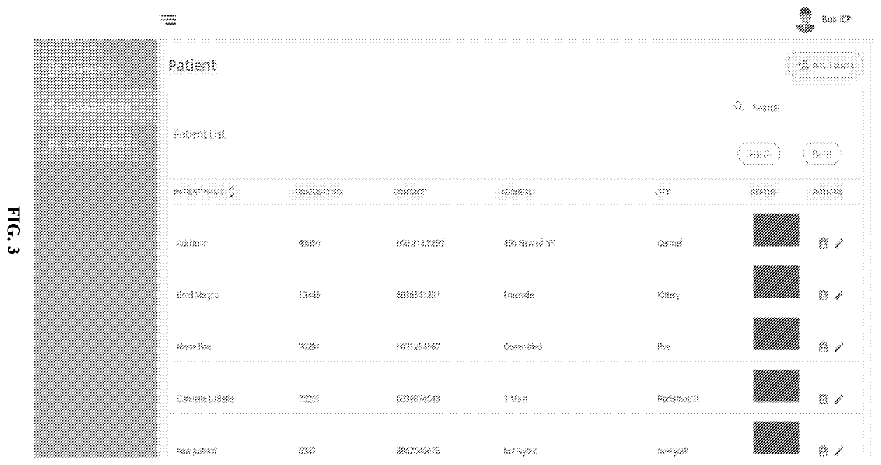

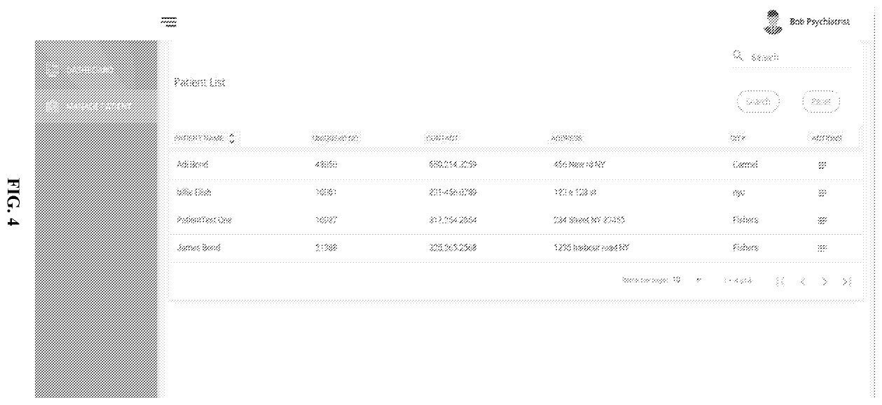

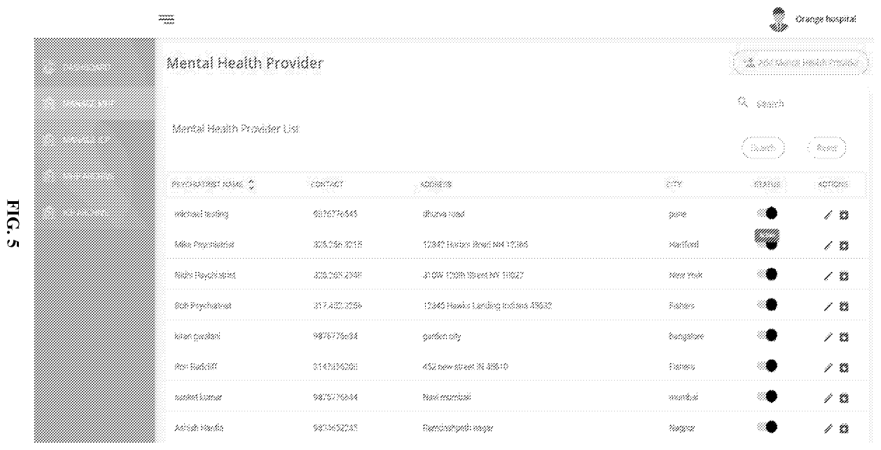

In some cases, a computing device can comprise graphical user interface (GUI). In some cases, the GUI can comprise a personal computing operating system for the computing device. The personal computing operating system can be a personal computer operating system, a mobile device operating system, a smart device operating system, another operating system or any combination thereof. In some cases, the GUI can comprise a virtual reality environment, a hologram or any combination thereof. A GUI can be configured to present the system described herein in a variety of ways. In some cases, the GUI can be configured to have one or more user interfaces. For example, the GUI can be configured to have a user interface for the subject and the GUI can be configured to have a user interface for a healthcare professional. This can be seen in, for example, FIG. 2, FIG. 3, FIG. 4, and FIG. 5. In some instances, the GUI can have the same interface for different users. The GUI can be configured to provide a subject's intent or a plurality of subject intents to a healthcare professional.

Questionnaire

The term “questionnaire,” as used herein, generally refers to a medical questionnaire that may be obtained from a subject. In some cases, a medical questionnaire can be an intake questionnaire. The questionnaire administered to a subject can comprise a single question or a plurality of questions. For example, the questionnaire may comprise about 2, 3, 4, 5, 6, 7, 8, 9, 10, 15, 20, 25, 30, 35, 40, 45, 50, 55, 60, 65, 70, 75, 80, 85, 90, 95, 100, or more than 100 questions. In some cases, the questionnaire can be an adaptable questionnaire. An adaptable questionnaire can comprise one or more questions that are selected by an AI-based algorithm (e.g., based at least in part on a previous response of the subject to one or more prior questions of the series of one or more questions). In some cases, a question can be directed to identify general subject information. For example, general information can be the subject's name, address, place of residence, current prescriptions, etc. In some cases, the question can be directed to assess the mental health of a subject. In some instances, a question can be derived from the American Psychological Association guidelines, The Diagnostic and Statistical Manual of Mental Disorders, a clinician, a mental health professional, an artificial intelligence program, or any combination thereof. In some cases, the question can be directed to assess the general health of a subject, for example, to determine their weight, any current illnesses, or a hearing issue. In some instances, a question can be binary or open-ended. A binary question can correspond to a “yes” or a “no” response from the subject. An open-ended question can have any response from the subject. In some cases, the question can be modified based on the age of the subject.

In some embodiments, a question can comprise an option to select a language from a plurality of languages for administering a series of one or more auditory questions. A question can comprise an option to select a language from a plurality of languages for administering a series of one or more visual questions. In some cases, a question can be stored on a database. In some instances, the database can comprise a cloud-based network, a local server, a remote server, or any combination thereof. In some instances, the database can comprise at least about: 10, 50, 100, 200, 300, 400, 500, 600, 700, 800, 900, 1000, 1500, 2000, 2500, 3000, 4000, 5000, 6000, 7000, 8000, 9000, 10000, 15000, or 20000 questions. In some instances, the database can comprise less than about: 10, 50, 100, 200, 300, 400, 500, 600, 700, 800, 900, 1000, 1500, 2000, 2500, 3000, 4000, 5000, 6000, 7000, 8000, 9000, 10000, 15000, or 20000 questions. In some instances, the database can comprise from about: 1 question to about 20000 questions, 1 question to about 50 questions, 10 questions to about 100 questions, 50 questions to about 300 questions, 100 questions to about 500 questions, 200 questions to about 750 questions, 400 questions to about 1000 questions, 750 questions to about 1500 questions, 1000 questions to about 3000 questions, 2000 questions to about 4000 questions, 3000 questions to about 7000 questions, 5000 questions to about 10000 questions, or about 10000 questions to about 25000 questions.

In some cases, the questionnaire may be obtained from a subject with a disease or disorder, from a subject that is suspected of having the disease or disorder, or from a subject that does not have or is not suspected of having the disease or disorder. The disease or disorder may be an infectious disease, an immune disorder or disease, a cancer, a genetic disease, a degenerative disease, a mental illness, disease, or disorder, a lifestyle disease, an injury, a rare disease, or an age-related disease. The infectious disease may be caused by bacteria, viruses, fungi, parasites or any combination thereof. The questionnaire may be taken before treatment of a subject. The questionnaire may be taken after treatment of a subject. The questionnaire may be taken during treatment of a subject. In some cases, the questionnaire can be given as a treatment. In some instances, the questionnaire may be given to a subject once. In some instances, the questionnaire may be given to the subject multiple times. Multiple questionnaires may be obtained from a subject to monitor the effects of a treatment over time. The questionnaire may be obtained from a subject having or suspected of having a disease or disorder for which a definitive positive or negative diagnosis is not available via clinical tests.

In some cases, a questionnaire can be administered at a health care facility. A health care facility can be a clinic, a free-clinic, an emergency room, a primary care facility, an urgent care facility, a mental health facility, a psychiatric ward, a school, a hospital, a detention facility, an inpatient facility or any combination thereof. In some instances, a health care facility can be a residential mental health treatment environment, a psychiatric residential center, a substance abuse rehabilitation center, a nursing home, an outpatient setting, a community mental health center, a mental health care providers office, a halfway house, or any combination thereof. In some cases, the questionnaire can be administered remotely. The questionnaire can be administered through a communications network. In some instances, a remote visit can be a telemedicine visit, a virtual visit, a virtual counseling session, a virtual group therapy session or any combination thereof. In some cases, a mental health professional review the questionnaire results at a health care facility. In some instances, the mental health professional can review the questionnaire results remotely. In some cases, a mental health professional can have a consultation with the subject. The consultation can be at the healthcare facility, a remote consultation (e.g., a telemedicine consultation), or any combination thereof.

In some embodiments, the questionnaire can be administered to a subject via visual intake, auditory intake, or any combination thereof. In some cases, the questionnaire can be auditorily presented to the subject by a speaker or a plurality of speakers that can be operable connected to one or more processors. The questionnaire can be administered by a female voice, a male voice, a child voice, an artificially generated (e.g., by a computer) voice, or any combination thereof. In some cases, the questionnaire may be obtained or derived from a recording from a human's voice. In some cases, the questionnaire can be visually presented to the subject on a graphical user interface (GUI), for example on a display such as a screen of a mobile device. In some cases, the GUI can be configured to present the questionnaire to the subject and provide the subjects intents to a health care professional. In some instances, the questionnaire is visually presented by text, a video, an image, or any combination thereof. The questionnaire can comprise visual pictures that provide information about a question to the subject. For example, the questionnaire can present a green symbol that indicates a “yes” answer or a red symbol that indicates a “no” answer.

In some cases, the visual, audio or any combination thereof questionnaire can be presented to a subject at least in part in English. In some cases, the questionnaire can be presented to a subject at least in part in a language other than English, such as one or more of: Spanish, Chinese, Mandarin Chinese, Hindi, Arabic, German, French, Arabic, Portuguese, Bengali, Russian, Japanese, Lahnda, Javanese, Korean, Telugu, Marathi, Turkish, Tamil, Vietnamese, or Urdu.

In some cases, the questionnaire can be responded to by the subject by an auditory, visual, kinesthetically, or any combination thereof response. In some instances, the responses from the questionnaire can be data that comprise visual data, audio data, or any combination thereof. In some cases, the response can comprise a subject's intent. The questionnaire can be responded to by a camera, a video camera, a microphone, a touch screen, a braille keyboard, a button, a switch, a stylus, a mouse, a keyboard or any combination thereof operably connected to one or more processors.

An auditory response can be a vocal response, for example, a spoken word or a plurality of spoken words. In some instances, a visual response can be a gesture, a movement, a facial expression, or any combination thereof. In some instances, a kinesthetic response can be a physical response, for example, a touch response. In some instances, the auditory response by a subject can be at least in part in English. In some instances, the auditory response by a subject can be at least in part in a language other than English, such as one or more of: Spanish, Chinese, Mandarin Chinese, Hindi, Arabic, German, French, Arabic, Portuguese, Bengali, Russian, Japanese, Lahnda, Javanese, Korean, Telugu, Marathi, Turkish, Tamil, Vietnamese or Urdu.

The visual, kinesthetic, or any combination thereof response by a subject can comprise an answer that can be at least in part in English. The visual, kinesthetic, or any combination thereof response by a subject can comprise an answer that can be at least in part in a language other than English, such as one or more of: Sign language, Spanish, Chinese, Mandarin Chinese, Hindi, Arabic, German, French, Arabic, Portuguese, Bengali, Russian, Japanese, Lahnda, Javanese, Korean, Telugu, Marathi, Turkish, Tamil, Vietnamese or Urdu. In some cases, the questionnaire may be responded to by providing no response.

Transmittal of Data

In some cases, data, subject intents, or any combination thereof obtained from the questionnaire can be transmitted through a network. The network can be a wired network, a wireless network, a cloud network, or any combination thereof. The data, subject intents, or any combination thereof can be transmitted through a secure method. In some instances, the transmitted information can be sent by a network to a second computing device. The second computing device can comprise a server, a cloud-based server, a personal computer, a smart mobile device or any combination thereof. The data, subject intents, or any combination thereof can be accessible by a health care professional during the administering of a medical questionnaire to the subject. In some instances, the information obtained from the questionnaire can be accessible by a health care professional after the administration of the medical questionnaire to the subject. The plurality of subject intents can be provided in a database format that can be assessable for a health care professional. The database format can be accessible inside the healthcare facility, remotely, or any combination thereof.

User Portals and Platforms

As used herein, a user can be a patient or a health care professional. In some cases, a patient can have mental health disorder. In some cases, a patient may not have a mental health disorder. In some cases, a health care professional can be a registered nurse, a physician, a physician's assistant, a dentist, doctor of osteopathic medicine, a midwife, a nurse practitioner, or any combination thereof. In some cases, a health care professional can be a psychiatrist, a clinical psychologist, an occupational therapist, a genetic counselor, a clinical social worker, a psychiatric-mental health nurse practitioner, a marriage and family therapist, a mental health therapist, a mental health counselor, a guidance counselor, a counselor, a therapist, or any combination thereof.

In an aspect, the present disclosure provides a system for generating a health profile of a subject. The health profile can comprise a mental health profile, a general health profile or an combination thereof. The system may comprise a database configured to store information obtained from the subject, which information comprises a plurality of subject intents, and one or more computer processors operatively coupled to the database. The one or more computer processors may be individually or collectively programmed to: (a) determine a subject intents, (b) record general information from the subject, (c) determine the general health of the subject, and (d) identify a potential health issue, a disease, a disorder or any combination thereof of the subject.

The system may generate the health profile of the subject (e.g., a mental health profile), facilitate data exchange of the mental health profile among end users (e.g., using a network such as a cloud network), store the mental health profile in a database (e.g., a cloud network), and/or display an electronic report comprising the mental health profile to an end user. In some instances, the electronic report can be an electronic health record.

The system may facilitate data exchange of the health profile among end users (e.g., using a network such as a cloud network). In some cases, the system may store the health profile in a database (e.g., a cloud network). The system may comprise a network interface that is in network communication with digital computers of different users. The network interface may include a portal or a platform, such as a user portal (e.g., for an end user to view mental health profiles) or a clinician portal (e.g., for a clinician to view or annotate mental health profiles). In some embodiments, a cloud-based method or system can be provided to a user for facilitating mental health data exchange. The user can use a web-application to log in and access his health data over a cloud-based computer system in the application, wherein the health data is generated from processing at least one question of the subject.

The systems and methods provided herein can include a user portal. In some instances, the user portal can comprise a user platform that can be configured to perform: health analysis; display health profiles and reports to a user; control access to health profiles, reports, data, or any combination thereof. The user portal, user platform or any combination thereof may include a server that can include a digital processing device or a processor that can execute machine code, such as a computer program or algorithm, to enable one or more method steps or operations, as disclosed herein. Such computer programs or algorithms can be run automatically or on-demand based on one or more inputs from the users. The user portal, user platform, or any combination thereof may allow users to connect with each other via the portal or platform, such as for mental health data exchange, thereby forming a network of connected users. Such data exchange can be secure and/or cloud-based. The users may each have an account for accessing the network and utilizing the functions associated with mental health data exchange securely and conveniently. The portal, platform, or any combination thereof may include a user interface, e.g., graphical user interface (GUI). The portal, platform, or any combination thereof may include a web application or mobile application. The portal, platform, or any combination thereof may include a digital display to display information to the user. In some cases, the portal, platform, or any combination thereof may include an input device that can interact with the user to accept input from the user.

In some embodiments, the electronic report comprising a subject's general information, general health information, mental health information or any combination thereof can be presented on a user interface, such as a graphical user interface (GUI), of an electronic device of a user (e.g., a healthcare professional). The electronic report may be transmitted to a remote user (e.g., a healthcare professional). Further, the electronic report can be stored on a remote server (e.g., a cloud-based server). In some cases, the electronic report can comprise an electronic medical record.

Referring to FIG. 2, this figure shows a patient's user interface that comprises a visually and auditorily administered question 201. The question is being presented by text and by sound. A patient is providing an auditorily presented answer to an administered question 202. The system records the answer from the patient and will select a new question from the database based on the previous answer or previous answers. The recorded answers to questions are analyzed and transcribed into a text format and presented to a medical professional. The system may also provide potential mental health issues determined by AI algorithms during the administration of the questionnaire. The potential mental health issues can be provided to a medical professional.

FIG. 3 shows an example of a screen shot from the application of an initial care provider (ICP) user interface. Referring to FIG. 3, this figure shows an initial care provider (ICP) user interface that comprises a patient name their contact information. The status of the questionnaire can be assessed from this view and selected for further analysis and treatment recommendations.

FIG. 4 shows an example of a screen shot from the application of a health care provider's user interface. Referring to FIG. 4, this figure shows a health care provider's user interface that comprises assigned patient names and corresponding contact information. The questionnaire can be selected from an assigned patient for further analysis by the healthcare provider.

FIG. 5 shows an example of a screen shot from the application of a hospital administrator's user interface. Referring to FIG. 5, this figure shows a hospital administrators user interface that comprises a psychiatrist name and their contact information. The completed or ongoing questionnaire can be sent to an available psychiatrist for analysis and treatment recommendations.

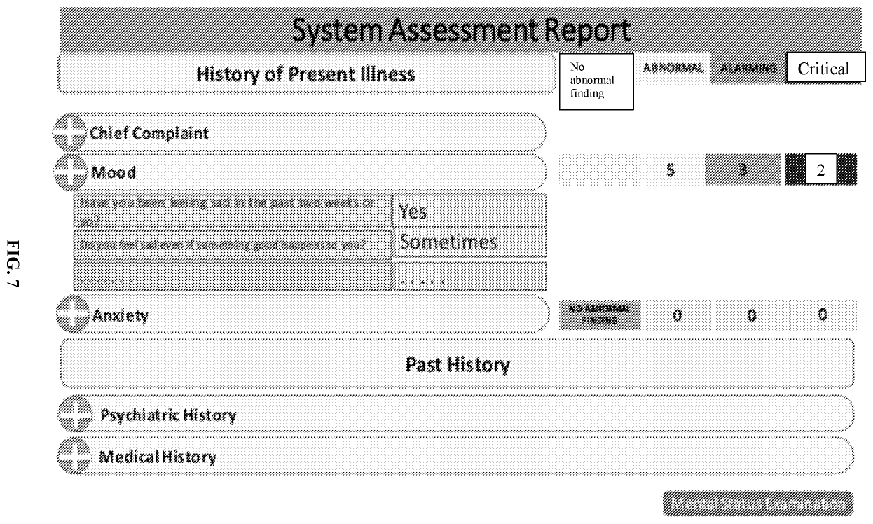

FIG. 7 shows an example of a screen shot from the system assessment report application of a health care provider's user interface. Referring to FIG. 7, this figure shows a health care provider's user interface that comprises the system assessment report. The system assessment report can provide information about the patient such as history of present illness, the chief complain, the mood, the anxiety and the medical history of the patient.

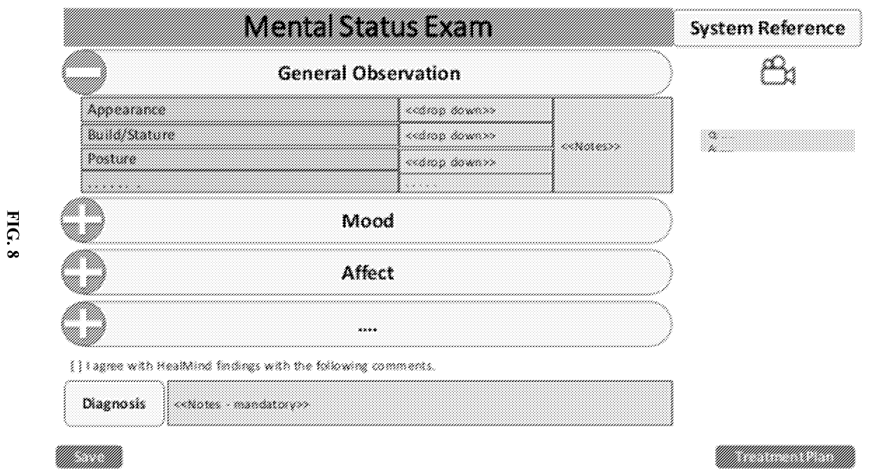

FIG. 8 shows an example of a screen shot from the mental status exam application of a health care provider's user interface. Referring to FIG. 8, this figure shows a health care provider's user interface that comprises the mental status exam. The mental status exam can provide information about the subject such as a general observation (appearance, build, posture), a subject's mood, and a diagnosis.

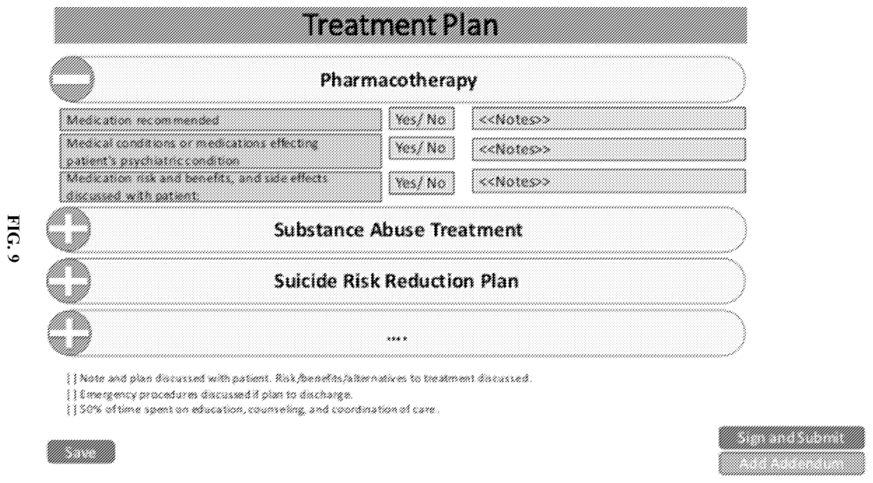

FIG. 9 shows an example of a screen shot from the treatment plan application of a health care provider's user interface. Referring to FIG. 9, this figure shows a health care provider's user interface that comprises the treatment plan application. The treatment plan application can provide treatment recommendations to a medical provider. A treatment recommendation can comprise a recommendation of a pharmacotherapy, a substance abuse treatment plan, a suicide risk reduction plan, or any combination thereof.

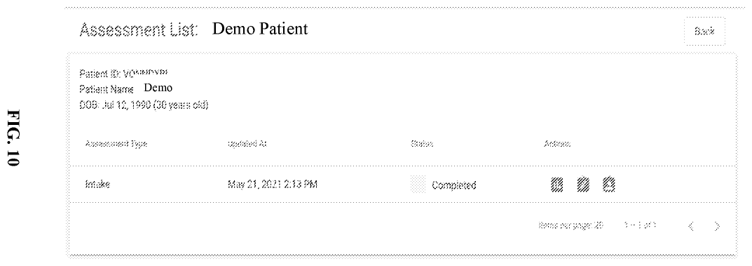

FIG. 10 shows an example of a screen shot from the application of a health care provider's user interface. Referring to FIG. 10, this figure shows a health care provider's user interface that comprises the status of the assessment taken by a subject. The assessment list can provide information on the assessment type and provide information on the status of the assessment (e.g. if the assessment has been completed or is in progress).

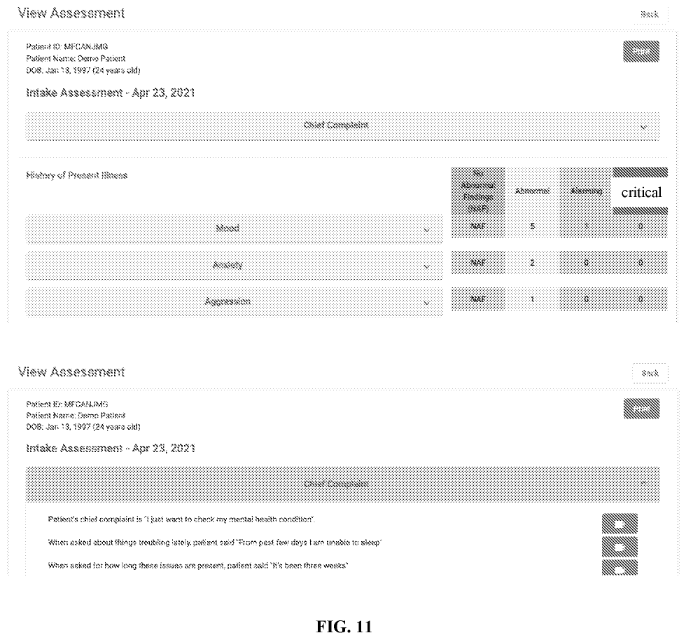

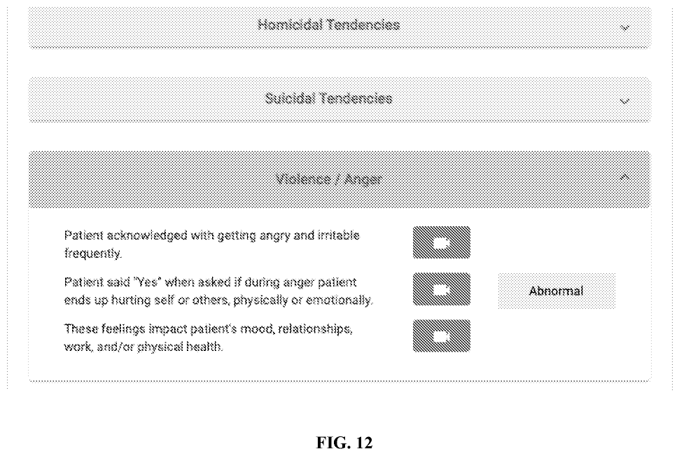

FIG. 11 and FIG. 12 shows an examples of a screen shots from the system assessment report application of a health care provider's user interface. Referring to FIG. 11, this figure shows a health care provider's user interface that comprises the system assessment report. The system assessment report can provide information about the patient such as history of present illness, the chief complaint, the mood, the anxiety, the aggression, and the medical history of the patient. FIG. 12 shows a health care provider's user interface that comprises the system assessment report that provides information about the patient's homicidal tendencies, suicidal tendencies, and violence and anger. The system assessment report can provide links to videos of the subject answering questions from an assessment.

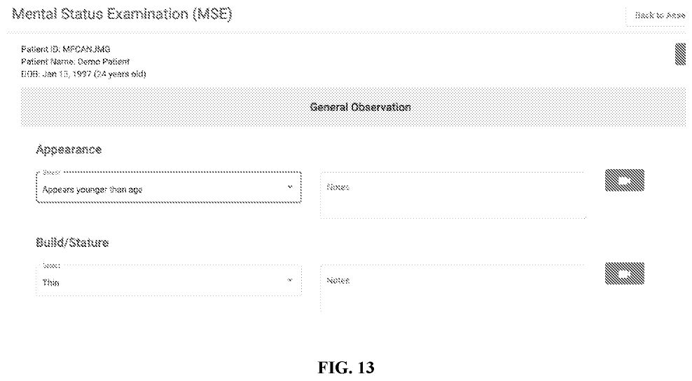

FIG. 13 shows an example of a screen shot from the mental status examination application of a health care provider's user interface. Referring to FIG. 13, this figure shows a health care provider's user interface that comprises a mental status examination application. This application allows a health care provider and/or the system to make notes on the general observation of a subject.

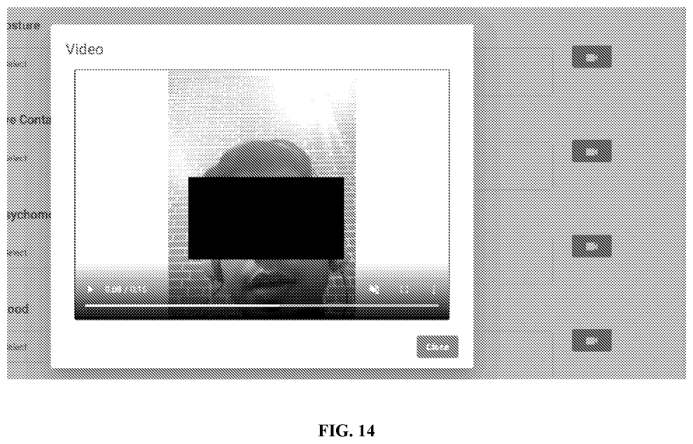

FIG. 14 shows an example of a screen shot of a video recording of a subject from the health care provider's user interface. Referring to FIG. 14, this figure shows a health care provider's user interface which shows a video recording of a subject answering a question from an assessment.

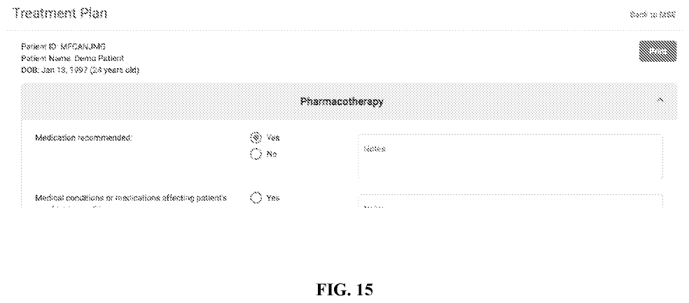

FIG. 15 shows an example of a screen shot from the treatment plan application of a health care provider's user interface. Referring to FIG. 15, this figure shows a health care provider's user interface that comprises the treatment plan application. The treatment plan application can provide treatment recommendations to a medical provider. A treatment recommendation can comprise a recommendation of a pharmacotherapy, a substance abuse treatment plan, a suicide risk reduction plan, or any combination thereof.

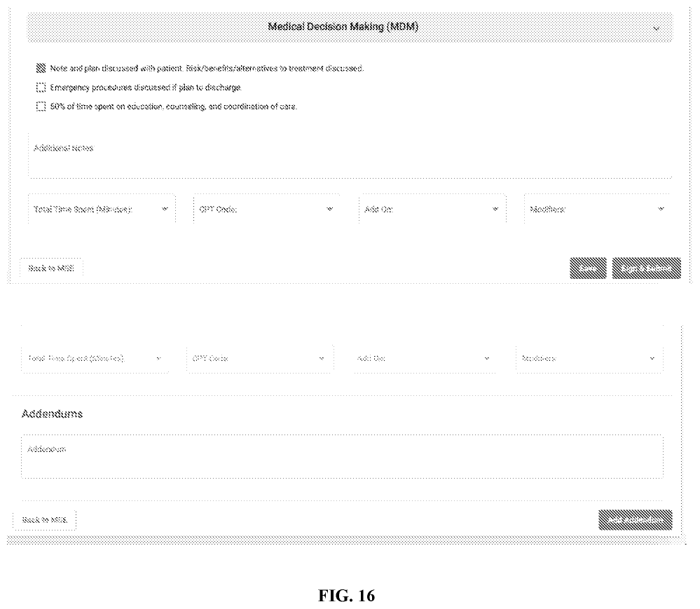

FIG. 16 shows an example of screen shots from the medical decision making application of a health care provider's user interface. Referring to FIG. 16, this figure shows a health care provider's user interface that comprises the medical decision making application. The medical decision making application can provide notes and/or instructions for medical decisions for a subject from a health care provider.

Artificial Intelligence

In some cases, an AI system can comprise an algorithm (e.g., a trained algorithm) or classifier as described herein. The AI system can be programmed to perform a logic-driven process (e.g., selecting a series of questions for an adaptable medical questionnaire). In some instances, the AI system can be programmed to analyze words and/or sentence structures from responses from a subject or a healthcare provider. For example, the AI system may be able to estimate the education level of a subject by the grammar of the responses from the subject. In some cases, the AI system can determine a summarized answer to an open-ended question. For example, if the subject has an answer that comprises over 500 words but at a basic level comprises the answer “yes”, or “affirmation” to a given question or scenario, the AI can determine the subject intent as “yes” and accordingly record the answer as “yes”. Similarly, if an answer comprises a broader response (e.g., “occasionally”, “sometimes”, “often”, or “of course”) the response can be processed by the AI to determine an intent (e.g., positive intent or negative intent) of the subject, and accordingly record the answer as affirmation or negation. In some cases, the AI can determine the intent of an answer by taking the last word, e.g., “Yes, I used to smoke, but not now” can be taken as no or negation for the smoking status.

In some cases, a yes or affirmation answer can comprise a word or words such as one or more: of course, yes, yeah, yup, yeah, yo, sometimes, i do, occasionally, constantly, okay, okiedokie, alright, ya, y′all, yoo-hoo, maybe, absolutely, indeed, certainly, some, I have to, I think, I think so, little bit, maybe, maybe maybe, ah I think so, oh, oh I think so, I think they believe me, I guess so, I think they were, usually, I am going to, what do you mean, I told you yes, always, always in my life, bit of both, a bit, every day, hell yeah, hahahaha yesssss, I am, mostly, most of the time, or any combination thereof.

In some cases, a no or negation answer can comprise a word or words such as one or more of: no, nah, never, not, not sure, no I, I don't, you kidding me, not a chance, nada, no way, negative, of course not, no way, not really, absolutely not, sometimes not, maybe not, certainly not, absolutely not, neither, I doubt, I don't think so, no I don't feel that, I don't feel so, I don't feel that, nada, nope, usually not, maybe not, of course no, I said I don't do it, I told you no, never, never ever, he dare not, dare not, I am not sure, I am not, she dare not, rarely, I guess no or any combination thereof. For example, an answer comprising “used to smoke” is no or negation for smoking status.

In some embodiments, the AI-based algorithm can comprise: a linear regression, a logistic regression, a Natural Language Processing (NLP), a Bayesian network, a maximum entropy, a conditional random field, a Random Forest, a support vector machine (SVM), a neural network, a deep learning algorithm, or a combination thereof.

In some cases, an AI algorithm can be programmed to determine potential health issues of a subject. In some instances, the AI algorithm can be programmed to determine a patient's risk to one or more health issues. A patient's risk can be separated into different categories. For example, a patient's risk can comprise the categories: normal, abnormal, alarming, and critical. In some instances, a patient can have a plurality of different patient risk categories. For example, a patient may be critical for a suicidal category and abnormal for an alcohol abuse category. In some cases, the different risk categories can be communicated to a healthcare professional through a communication medium. In some instances, the risk can be determined after completion of the questionnaire. In some instances, the risk can be determined during the questionnaire. For example, if a patient has a critical alarm for a suicidal category the AI can alert the healthcare professional during the administration of the questionnaire.

In some cases, an AI algorithm can comprise transcribing the data from the responses from the subject. The data can be transcribed from visual data, audio data, physically inputted data (e.g., a touchscreen or keyboard selected answer), or any combination thereof. In some cases, the transcribing can be at least in part in English. The transcribing can be performed in a language primarily other than English. For example, the transcribing can be in Chinese but may contain an English word for which there may not be a Chinese equivalent. In some cases, the transcribing can be at least in part in: Spanish, Chinese, Mandarin Chinese, Hindi, Arabic, German, French, Arabic, Portuguese, Bengali, Russian, Japanese, Lahnda, Javanese, Korean, Telugu, Marathi, Turkish, Tamil, Vietnamese or Urdu. In some cases, the data can be translated prior to transcribing. In some instances, the translation can be at least in part to English. The translation can be at least in part to a language primarily other than English. For example, the translation can be in Spanish but may contain an English word for which there may not be a Spanish equivalent. In some cases, the translation can be at least in part to a language selected from Spanish, Chinese, Mandarin Chinese, Hindi, Arabic, German, French, Arabic, Portuguese, Bengali, Russian, Japanese, Lahnda, Javanese, Korean, Telugu, Marathi, Turkish, Tamil, Vietnamese, or Urdu.

In some cases, the transcribing of a series of one or more responses from a subject into the plurality of subject intents can comprise selecting a subject intent from among a plurality of subject intents. For example, the artificial intelligence system described herein can select a subject's intent from a single response or can determine a subject's intent from multiple responses. In some cases, the responses may be from a directly related question or may be from an indirectly related question. In some cases, the plurality of subject intents can comprise binary subject intents.

In some cases, the one or more processors can be configured to execute the one or more executable instructions to further identify a set of one or more potential health issues based at least in part on the plurality of subject intents. In some instances, the AI can be programmed to further identify a set of one or more potential health issues based at least in part on the plurality of subject intents. In some cases, the system described herein, can be configured to generate a diagnosis or an indication of a subject having a disease, disorder or being at an elevated risk for a disease or a disorder. For example, the system can be configured to generate a mental health diagnosis such as depression, or anxiety. In some instances, the system can be configured to provide the health issues of the subject to a health care professional. In some cases, a subject intents, a subject's data, a potential health issue, a disease, a disorder, or any combination thereof can be provided to a health care professional through the GUI.

The artificial intelligence system described herein can comprise parametric models (e.g., linear regression models, logistic regression models, quantile regression models, support vectors machines (SVMs), random forest models, boosted tree models, etc.) that can be used to predict parameters (e.g., a median, a mean, a mode, a variance, a standard deviation, a quantile, a measure of central tendency, a measure of variance, a range, a minimum, a maximum, an interquartile range, a percentile, or a combination thereof) of the questionnaire variables. As one example, regression models may be trained to learn from historical data how these responses vary over time, based on features such as a response to a frequency of drinking question, etc. The trained regression models may then be used to predict the prior and conditional distribution of the random variables. The parametric models can be used to select a new question. In some cases, the parametric models can be used to determine a subject's intent based on one or more responses from a subject.

Alternatively, the system may comprise non-parametric models such as Gaussian processes, Dirichlet processes, or other Bayesian sampling-based techniques. A Gaussian process is a stochastic model defined by a collection of random variables indexed by time. Each finite collection of the random variables has a multivariate normal distribution. That is, each finite collection of the random variables is normally distributed. The distribution of a Gaussian process is the joint distribution of all the random variables, and as such, it is a distribution over functions with a continuous domain. A Dirichlet process, meanwhile, is a stochastic model whose range itself is a set of probability distributions.

In some cases, the artificial intelligence comprises a classifier (e.g., a machine learning classifier). The classifier can be programmed to create an adaptable questionnaire. In some instances, the classifier may be programmed to classify one or more responses of the mental health questions as corresponding to one or more mental health states of the subject. In some cases, the classifier can comprise a natural language processing system. In some instances, the classifier can comprise a neural network, a deep learning algorithm, a Bayesian Network, a maximum entropy, a conditional random field or any combination thereof. The classifier may comprise a supervised machine learning algorithm or an unsupervised machine learning algorithm. The classifier may comprise a classification and regression tree (CART) algorithm. The classifier may comprise, for example, a support vector machine (SVM), a linear regression, a logistic regression, a nonlinear regression, a neural network, a Random Forest, a deep learning algorithm, a naïve Bayes classifier. The classifier may comprise an unsupervised machine learning algorithm, e.g., clustering analysis (e.g., k-means clustering, hierarchical clustering, mixture models, DBSCAN, OPTICS algorithm), principal component analysis, independent component analysis, non-negative matrix factorization, singular value decomposition, anomaly detection (e.g., local outlier factor), neural network (e.g., autoencoder, deep belief network, Hebbian learning, generative adversarial network, self-organizing map), expectation-maximization algorithm, and method of moments.

The classifier may be configured to accept a plurality of input variables and to produce one or more output values based on the plurality of input variables. The plurality of input variables may comprise data indicative of a set of identified mental health issues. For example, an input variable may comprise a set of identified mental health disorders. As another example, the plurality of input variables may comprise one or more responses of the mental health questions.

The classifier may have one or more possible output values, each comprising one of a fixed number of possible values (e.g., a linear classifier, a logistic regression classifier, etc.) indicating a classification of the one or more responses of the mental health questions into an assessment of a mental health state (e.g., severity of mental illness, such as a depression level of the subject). The classifier may comprise a binary classifier, such that each of the one or more output values comprises one of two values (e.g., {0, 1}, {positive, negative}, {high impact, low impact}, {high risk, low risk}, or {diseased, non-diseased}) indicating a classification of the one or more responses of the mental health questions into an assessment of a mental health state. The classifier may be another type of classifier, such that each of the one or more output values comprises one of more than two values (e.g., {0, 1, 2}, {positive, negative, or indeterminate}, {beneficial impact, low impact, medium impact, high impact, and/or very high impact}, {high risk, medium risk, low risk, zero risk}, or {diseased, non-diseased, or indeterminate}) indicating a classification of the one or more responses of the mental health questions into an assessment of a mental health state (e.g., severity of mental illness).

The output values may comprise descriptive labels, numerical values, or a combination thereof. Some of the output values may comprise descriptive labels. Such descriptive labels may provide an identification or indication of the mental health (e.g., severity of mental illness) of the subject, and may comprise, for example, normal, abnormal, alarming, critical or any combination thereof. Such descriptive labels may provide an identification of a recommendation for the subject's mental health and may comprise, for example, a behavioral change, stress management, a therapeutic intervention, a duration of the therapeutic intervention, a recommendation related to diet, exercise, sports training, supplements, functional tests, blood tests, brain management, skin care, and environmental exposure. Such descriptive labels may provide an identification of secondary clinical tests that may be appropriate to perform on the subject, and may comprise, for example, a biopsy, a blood test, a functional test, a psychological examination, a computed tomography (CT) scan, a magnetic resonance imaging (MRI) scan, an ultrasound scan, a chest X-ray, a positron emission tomography (PET) scan, or a PET-CT scan. Such descriptive labels may provide a prognosis of the disease state of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}. Such integer output values may comprise, for example, {0, 1, 2}. Such continuous output values may comprise, for example, a probability value of at least 0 and no more than 1 (e.g., of the classification of the one or more responses of the mental health questions into assessment of a mental health state). Such continuous output values may comprise, for example, an un-normalized probability value of at least 0. Such continuous output values may comprise, for example, an un-normalized probability value of at least 0. Such continuous output values may comprise, for example, an indication of an expected duration of an intervention, or a severity of mental illness, such as a depression level of the subject. Some numerical values may be mapped to descriptive labels, for example, by mapping 1 to “positive” and 0 to “negative.”

Some of the output values may be assigned based on one or more cutoff values. For example, a binary classification of one or more responses of the mental health questions may assign an output value of “positive” or 1 if the question response indicates that the subject has at least a 50% probability of being recommended an intervention or being diagnosed with a mental health disorder as a result of the mental health question. For example, a binary classification of question responses may assign an output value of “negative” or 0 if the question response indicates that the subject has less than a 50% probability of being recommended an intervention or being diagnosed with a mental health disorder as a result of the mental health question. In this case, a single cutoff value of 50% is used to classify question responses into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, and about 99%.

As another example, a classification of one or more responses of the mental health questions may assign an output value of “positive” or 1 if the question response indicates that the subject has a probability of being recommended an intervention or being diagnosed with a mental health disorder of at least 50%, at least about 55%, at least about 60%, at least about 65%, at least about 70%, at least about 75%, at least about 80%, at least about 85%, at least about 90%, at least about 95%, at least about 98%, or at least about 99%. The classification of one or more responses of the mental health questions may assign an output value of “positive” or 1 if the question response indicates that the subject has a probability of being recommended an intervention or being diagnosed with a mental health disorder of more than 50%, more than 55%, more than 60%, more than 65%, more than 70%, more than 75%, more than 80%, more than 85%, more than 90%, more than 95%, more than 98%, or more than 99%.

The classification of one or more responses of the mental health questions may assign an output value of “negative impact” or 0 if the question response indicates that the subject has a probability of being recommended an intervention or being diagnosed with a mental health disorder of less than 50%, less than 45%, less than 40%, less than 35%, less than 30%, less than 25%, less than 20%, less than 10%, less than 5%, less than 2%, or less than 1%. The classification of one or more responses of the mental health questions may assign an output value of “negative” or 0 if the question response indicates that the subject has a probability of being recommended an intervention or being diagnosed with a mental health disorder of no more than 50%, no more than 45%, no more than 40%, no more than 35%, no more than 30%, no more than 25%, no more than 20%, no more than 10%, no more than 5%, no more than 2%, or no more than 1%.

The classification of one or more responses of the mental health questions may assign an output value of “indeterminate” or 2 if the one or more responses of the mental health questions have not been classified as “positive,” “negative,” 1, or 0. In this case, a set of two cutoff values is used to classify one or more responses of the mental health questions into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify one or more responses of the mental health questions into one of n+1 possible output values, where n is any positive integer.

The classifier may be trained with a plurality of independent training samples of a training dataset comprising one or more responses of the mental health questions. Each of the independent training samples of the of a training dataset may comprise one or more responses of the mental health questions from a subject, associated data obtained by processing the one or more responses of the mental health questions (as described elsewhere herein), and one or more known output values corresponding to classifier outputs, such as mental health states of the subject. Independent training samples of a training dataset may comprise one or more responses of the mental health questions and associated data and outputs obtained from a plurality of different subjects. Independent training samples may comprise one or more responses of the mental health questions and associated data and outputs obtained at a plurality of different time points from the same subject. Independent training samples may be associated with presence of a mental health state (e.g., training samples comprising responses of the mental health questions and associated data and outputs obtained from a plurality of subjects known to have the mental health state). Independent training samples may be associated with absence of a mental health state (e.g., training samples comprising responses of the mental health questions and associated data and outputs obtained from a plurality of subjects who are known to not have the mental health state).

The classifier may be trained with at least about 50, at least about 100, at least about 150, at least about 200, at least about 250, at least about 300, at least about 350, at least about 400, at least about 450, or at least about 500 independent training samples. The independent training samples may comprise samples associated with presence of the mental health state and/or samples associated with absence of the mental health state. The classifier may be trained with no more than 500, no more than 450, no more than 400, no more than 350, no more than 300, no more than 250, no more than 200, no more than 150, no more than 100, or no more than 50 independent training samples associated with presence of the mental health state. In some embodiments, the given test sample is independent of training samples used to train the classifier.

The classifier may be trained with a first number of independent training samples associated with a presence of the mental health state and a second number of independent training samples associated with an absence of the mental health state. The first number of independent training samples associated with a presence of the mental health state may be no more than the second number of independent training samples associated with an absence of the mental health state. The first number of independent training samples associated with a presence of the mental health state may be equal to the second number of independent training samples associated with an absence of the mental health state. The first number of independent training samples associated with a presence of the mental health state may be greater than the second number of independent training samples associated with an absence of the mental health state.

The classifier may be configured to identify the mental health state with an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%; for at least about 50, at least about 100, at least about 150, at least about 200, at least about 250, at least about 300, or more than about 300 independent training samples. The accuracy of identifying the mental health state by the classifier may be calculated as the percentage of independent test samples (e.g., subjects having the mental health state) that are correctly identified or classified as having or not having the mental health state, respectively.

The classifier may be configured to identify the mental health state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The PPV of identifying the mental health state by the classifier may be calculated as the percentage of independent test samples identified or classified as having the mental health state that correspond to subjects that truly have the mental health state. A PPV may also be referred to as a precision.

The classifier may be configured to identify the mental health state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The NPV of identifying the mental health state by the classifier may be calculated as the percentage of independent test samples identified or classified as not having the mental health state that correspond to subjects that truly do not have the mental health state.

The classifier may be configured to identify the mental health state with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The clinical sensitivity of identifying the mental health state by the classifier may be calculated as the percentage of independent test samples associated with presence of the mental health state (e.g., subjects known to have the mental health state) that are correctly identified or classified as having the mental health state. A clinical sensitivity may also be referred to as a recall.

The classifier may be configured to identify the mental health state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The clinical specificity of identifying the mental health state by the classifier may be calculated as the percentage of independent test samples associated with absence of the mental health state (e.g., apparently healthy subjects with negative clinical test results for the mental health state) that are correctly identified or classified as not having the mental health state.

The classifier may be configured to identify the mental health state with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The AUC may be calculated as an integral of the Receiver Operator Characteristic (ROC) curve (e.g., the area under the ROC curve) associated with the classifier in classifying independent test samples as having or not having the mental health state.

The classifier may be adjusted or tuned to improve the performance, accuracy, PPV, NPV, clinical sensitivity, clinical specificity, or AUC of identifying one or more mental health states. The classifier may be adjusted or tuned by adjusting parameters of the classifier (e.g., a set of cutoff values used to classify a sample as described elsewhere herein, or weights of a neural network). The classifier may be adjusted or tuned continuously during the training process or after the training process has completed.

After the classifier is initially trained, a subset of the inputs may be identified as most influential or most important to be included for making high-quality classifications. For example, a subset of the input variables may be identified as most influential or most important to be included for making high-quality classifications or identifications of the mental health state. The set of input variables or a subset thereof may be ranked based on metrics indicative of each input variable's influence or importance toward making high-quality classifications or identifications of the mental health state. Such metrics may be used to reduce, in some cases significantly, the number of input variables (e.g., predictor variables) that may be used to train the classifier to a desired performance level (e.g., based on a desired minimum accuracy, PPV, NPV, clinical sensitivity, clinical specificity, or AUC).

For example, if training the training algorithm with a plurality comprising several dozen or hundreds of input variables (e.g., responses to questions) in the classifier results in an accuracy of classification of more than 99%, then training the classifier instead with only a selected subset of 1, 2, 3, 4, 5, 6, 7, 8, 9, 10, no more than about 10, no more than about 15, no more than about 20, no more than about 25, no more than about 30, no more than about 35, no more than about 40, no more than about 45, no more than about 50, or no more than about 100 such most influential or most important input variables (e.g., responses to questions) among the plurality results in decreased but still acceptable accuracy of classification (e.g., at least about 70%, at least about 75%, at least about 80%, at least about 85%, at least about 90%, at least about 95%, at least about 96%, at least about 97%, or at least about 98%).

In some embodiments, the subset may be selected by rank-ordering the entire plurality of input variables (e.g., responses to questions) and selecting a predetermined number (e.g., 1, 2, 3, 4, 5, 6, 7, 8, 9, 10, no more than about 10, no more than about 15, no more than about 20, no more than about 25, no more than about 30, no more than about 35, no more than about 40, no more than about 45, no more than about 50, no more than about 100, no more than about 150, or no more than about 200) of input variables with the best metrics. This subset of input variables may be used as part of an adaptable medical questionnaire.

Computer Systems

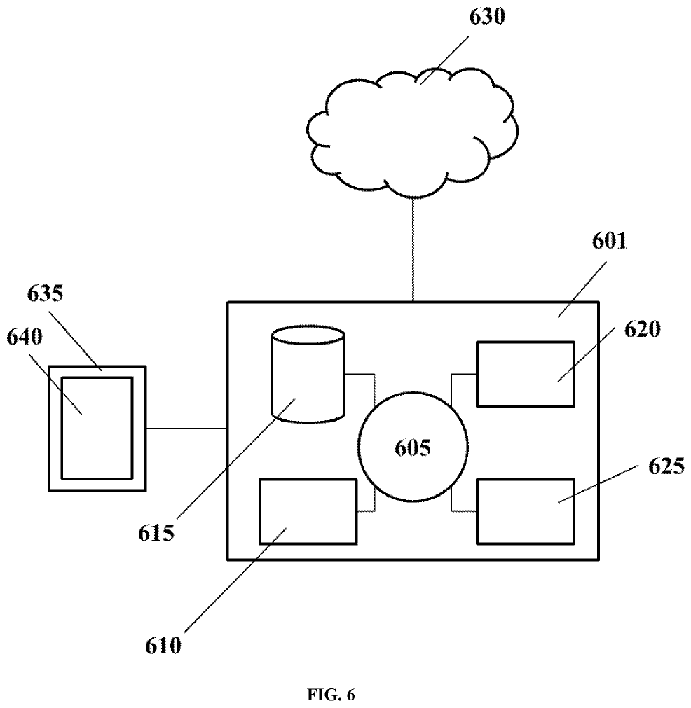

The present disclosure provides computer systems that are programmed to implement methods of the disclosure. FIG. 6 shows a computer system 601 that is programmed or otherwise configured to perform one or more functions or operations for facilitating mental health reporting for a subject. The computer system 601 can regulate various aspects of the portal and/or platform of the present disclosure, such as, for example, retrieving a medical questionnaire for a subject, generating an adaptable medical questionnaire comprising a series of one or more medical questions to be presented to the subject, presenting the adaptable medical questionnaire to the subject, receiving data of a series of one or more responses from the subject, responsive to the presenting, and processing the data to transcribe the series of one or more responses from the subject into a plurality of subject intents. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet.

The network 630 in some cases is a telecommunication and/or data network. The network 630 can include one or more computer servers, which can enable distributed computing, such as cloud computing. For example, one or more computer servers may enable cloud computing over the network 630 (“the cloud”) to perform various aspects of analysis, calculation, and generation of the present disclosure, such as, for example, retrieving a medical questionnaire for a subject, generating an adaptable medical questionnaire comprising a series of one or more medical questions to be presented to the subject, presenting the adaptable medical questionnaire to the subject, receiving data of a series of one or more responses from the subject, responsive to the presenting, and processing the data to transcribe the series of one or more responses from the subject into a plurality of subject intents. Such cloud computing may be provided by cloud computing platforms such as, for example, Amazon Web Services (AWS), Microsoft Azure, Google Cloud Platform, and IBM cloud. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC). The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user (e.g., a mobile device of the user). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods provided herein can be implemented by way of machine (e.g., computer processor) executable code stored on an electronic storage location of the computer system 601, such as, for example, on the memory 610 or electronic storage unit 615. The machine-executable or machine-readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 601 can include or be in communication with an electronic display 635 that comprises a user interface (UI) 640 for providing, for example, questions or other data management. Examples of user interfaces include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, retrieving a medical questionnaire for a subject, generating an adaptable medical questionnaire comprising a series of one or more medical questions to be presented to the subject, presenting the adaptable medical questionnaire to the subject, receiving data of a series of one or more responses from the subject, responsive to the presenting, and processing the data to transcribe the series of one or more responses from the subject into a plurality of subject intents.

### EXAMPLES

### Example 1: Determining a Mental Health Profile of a Patient by Administering an Intake Mental Health Questionnaire

Using the systems and methods of the present disclosure, a mental health profile can be generated from a subject at least in part by the subject answering a mental health questionnaire. First, a system is developed and trained with an adaptable mental health intake questionnaire. The adaptable intake questionnaire is developed with artificial intelligence to select future questions. The intake questionnaire is presented by a mobile device (e.g., a tablet computer) to a patient prior to the patient meeting with a healthcare professional. The questionnaire is administered by audio presentation via a speaker and text presentation via the patient GUI. The patient responds to the questionnaire verbally and the system records the patient's answers. Next, the system is developed to transcribe, translate, and evaluate the answers provided by a patient from the questionnaire and determines potential mental health risks. The mental health risks can be evaluations from mental health disorders, such as substance-related and addictive disorder, a non-substance-related disorder, an anxiety disorder, an attention deficit disorder, an attention hyperactivity disorder, bipolar and related disorders, a depressive disorders, an eating disorder, obsessive compulsive and related disorder, schizophrenia spectrum and other psychotic disorders, dissociative disorders, somatic-symptom and related disorders, sexual disorders, sleep-wake disorders, personality disorders, trauma and stress disorders, neuro-cognitive disorders, disruptive impulse-control and conduct disorders, other mental disorders or any combination thereof. The system may determine additional information such as the degree of risk, for example, normal, abnormal, alarming, and critical. Next, the information is provided to a mental health professional via a secured wired or wireless connection. The mental health professional can view the potential mental health risks, and transcribed answers via the health care providers user interface on the GUI to evaluate potential therapies, treatments, or any combination thereof.

### Example 2: AI-Assisted Digital Technology Platform

Using the systems and methods of the present disclosure, an AI-assisted digital technology platform is used to improve delivery of behavioral health care services to patients. Previous patient workflows may face numerous challenges throughout the process of accepting a patient, assessing the patient, and admitting or triaging the patient. For example, accepting the patient may involve the patient visiting the emergency department with a mental health complaint, and experiencing an excessive wait time if the emergency department does not have a psychiatrist on duty, thereby leading to loss of revenue for the emergency department. As another example, assessing the patient may involve significant time needed for assessment and limit treatment to only a few patients per day, thereby leading to suboptimal patient care. As another example, the triage process may be fully dependent on the physical presence of a psychiatrist at the emergency department, thereby leading to limit opportunities to treat patients.

The improved patient workflow may comprise improved methods of accepting a patient, assessing the patient, and admitting or triaging the patient. For example, accepting the patient may involve accepting the patient even without an on-duty psychiatrist present. The initial care provider (ICP) logs into a web-based system to create a patient profile (e.g., with optional electronic health record integration). The patient agrees to the terms (e.g., which are HIPPA compliant), and the ICP initiates an assessment on a digital assistant (e.g., an iPad) at the kiosk. As another example, assessing the patient may involve the patient taking an assessment (e.g., medical questionnaire) with the iPad. The touch-free, fully voice-based conversation allows the patient to express himself or herself freely. The emergency department can handle multiple patient assessments simultaneously with a single psychiatrist physical present or not. The real-time audio and video recording and transcription of the full assessment are recorded and made available to a health care provider, such as a psychiatrist (either immediately or stored for retrieval at a later time, thereby resulting in improved patient care. As another example, the triage process may involve the psychiatrist reviewing the assessment remotely. The VTC (telemedicine) consultation can be executed in real time, and the psychiatrist recommends a treatment plan. The ICP executes the triage process based on instructions from the psychiatrist. Therefore, more patients are able to be treated in less time, with higher efficacy, effectiveness, and patient and provider satisfaction.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A system comprising:
a computing device comprising a memory, one or more executable instructions, one or more processors operably connected to the memory, wherein the one or more processors are configured to execute the one or more executable instructions to perform at least:
(a) retrieving a medical questionnaire for a subject;
(b) generating an adaptable medical questionnaire comprising a series of one or more medical questions to be presented to the subject, wherein subsequent questions of the series of one or more medical questions are selected by an artificial intelligence (AI)-based algorithm based at least in part on a previous response of the subject to one or more prior questions of the series of one or more medical questions;
(c) presenting the adaptable medical questionnaire to the subject;
(d) receiving data of a series of one or more responses from the subject, responsive to the presenting
(e) processing the data to transcribe the series of one or more responses from the subject into a plurality of subject intents, wherein at least one of the processing and selecting the subsequent questions are performed in real-time; and
(f) generating an indication of a subject having a disease or disorder or being at elevated risk for a disease or disorder, wherein the disease or disorder is selected from the group consisting of: a substance-related and addictive disorder, a non-substance-related disorder, an anxiety disorder, an attention deficit disorder, an attention hyperactivity disorder, a bipolar disorder, a depressive disorders, an eating disorder, an obsessive compulsive disorder, a schizophrenia disorder, a psychotic disorder, a dissociative disorder, a somatic-symptom disorder, a sexual disorder, a sleep-wake disorder, a personality disorder, a trauma and stress disorder, a neuro-cognitive disorder, a disruptive impulse-control and conduct disorder, a mental disorders, and any combination thereof.

2. The system of claim 1, wherein the computing device is a mobile device.

3. The system of claim 2, wherein the mobile device comprises a tablet computer, a laptop computer, a smartphone, or a smartwatch.

4.-5. (canceled)

6. The system of claim 1, further comprising a camera operably connected to the one or more processors, a microphone and a speaker operably connected to the one or more processors, or both.

7. (canceled)

8. The system of claim 1, further comprising a graphical user interface (GUI) and wherein the GUI is configured to perform at least one of: presenting the adaptable medical questionnaire to the subject and providing the plurality of subject intents to a health care professional.

9.-13. (canceled)

14. The system of claim 8, wherein the GUI comprises a first user interface to be used by the subject, and wherein the GUI comprises a second user interface to be used by the health care professional.

15.-17. (canceled)

18. The system of claim 1, wherein the adaptable medical questionnaire is administered to the subject to be answered prior to or during a health care visit of the subject.

19. The system of claim 1, wherein the adaptable medical questionnaire is administered via one or more of auditory intake and visual intake.

20.-35. (canceled)

36. The system of claim 1, wherein the medical questionnaire is retrieved from a database.

37. (canceled)

38. The system of claim 1, wherein the series of one or more medical questions comprises medical questions for assessing mental health.

39.-40. (canceled)

41. The system of claim 1, wherein the medical questionnaire comprises medical questions derived from American Psychological Association (APA) guidelines.

42. The system of claim 36, wherein the database comprises at least about 10, about 50, about 100, about 200, about 300, about 400, about 500, about 600, about 700, about 800, about 900, about 1000, about 1500, about 2000, about 2500, or about 3000 medical questions.

43. The system of claim 42, wherein the medical questions comprise binary or open-ended questions.

44. (canceled)

45. The system of claim 43, comprising the open-ended questions which comprise open-ended questions corresponding to open-ended responses from the subject.

46. (canceled)

47. The system of claim 1, wherein the adaptable medical questionnaire is administered remotely to the subject through a communications network.

48. The system of claim 1, wherein the subject is a patient.

49.-52. (canceled)

53. The system of claim 1, wherein the subsequent questions comprise questions generated by an AI-based algorithm.

54.-58. (canceled)

59. The system of claim 1, wherein the AI-based algorithm is programmed to determine a patient risk of one or more potential health issues, wherein patient risk is determined from among a plurality of different patient risk categories, and wherein the plurality of different patient risk categories comprises at least two of: critical, alarming, abnormal, and normal.

60.-63. (canceled)

64. The system of claim 1, wherein the transcribing is performed at least in part in English, or in a language primarily other than English.

65.-69. (canceled)

70. The system of claim 1, wherein transcribing the series of one or more responses from the subject into the plurality of subject intents comprises selecting a subject intent from among a plurality of subject intents, and wherein the plurality of subject intents comprise binary subject intents.

71.-89. (canceled)
